(19) United States
(12) Patent Application Publication
(10) Pub. No.: US 2025/0181903 A1
(43) Pub. Date: Jun. 5, 2025

(54) NEURAL INTERFACE DEVICE

(71) Applicant: Science Corporation, Alameda, CA (US)

(72) Inventors: Max Hodak, Alameda, CA (US); Alan Mardinly, Alameda, CA (US); Yifan Kong, Alameda, CA (US)

(73) Assignee: Science Corporation, Alameda, CA (US)

(21) Appl. No.: 18/970,461

(22) Filed: Dec. 5, 2024

Related U.S. Application Data
(60) Provisional application No. 63/606,381, filed on Dec. 5, 2023.

(51) Int. Cl. G06N 3/06

(52) U.S. Cl. CPC G06N 3/061

(57) ABSTRACT

In variants, the system can include: a controller, a set of scaffolds, and a tissue interface. The system can optionally include and/or be used with an external device. In a specific example, the system can be or include an intracortical brain-computer interface that can be embedded in-situ on the surface of the brain.

### CROSS-REFERENCE TO RELATED APPLICATIONS

This application claims the benefit of U.S. Provisional Application No. 63/606,381 filed 5 Dec. 2023, which is incorporated in its entirety by this reference.

This application is related to U.S. application Ser. No. 18/740,854 filed 12 Jun. 2024, which is a continuation of U.S. application Ser. No. 18/219,366 filed 7 Jul. 2023, which claims the benefit of U.S. Provisional Application No. 63/359,100 filed 7 Jul. 2022, U.S. Provisional Application No. 63/433,130 filed 16 Dec. 2022, and U.S. Provisional Application No. 63/438,947 filed 13 Jan. 2023, each of which is incorporated in its entirety by this reference.

### TECHNICAL FIELD

This invention relates generally to the brain-computer interface field, and more specifically to a new and useful system and method in the brain-computer interface field.

### BRIEF DESCRIPTION OF THE FIGURES

FIG. 1 is a schematic representation of a variant of the system.

FIG. 2 is a schematic representation of an example of the system.

FIG. 3A depicts a specific example of a proximal subsystem, including a set of scaffolds, a set of spacers, and a tissue interface.

FIG. 3B depicts a specific example of a proximal subsystem, including a scaffold connector connecting scaffolds via conductive elements (e.g., copper bumps).

FIG. 4 depicts an example of a proximal subsystem anchored to a tissue of interest.

FIG. 5A depicts an example of a proximal subsystem anchored to a tissue of interest and a distal subsystem anchored to a skull, where the distal subsystem is exposed.

FIG. 5B depicts another example of a proximal subsystem anchored to a tissue of interest and a distal subsystem anchored to a skull, where the distal subsystem is covered by skin.

FIG. 6A depicts an example of a set of scaffolds with spacers between pairs of scaffolds.

FIG. 6B depicts an example of a set of scaffolds with a scaffold connector connecting scaffolds via conductive elements (e.g., copper bumps).

FIG. 7A depicts a specific example of a set of scaffolds, showing the left side of each scaffold, showing the left side of each scaffold containing an array of excitation elements (e.g., μLEDs).

FIG. 7B depicts a specific example of a set of scaffolds (e.g., the set of scaffolds shown in FIG. 7A), showing the right side of each scaffold containing an array of sensor elements (e.g., electrodes).

FIG. 8 depicts an illustrative example of a proximal subsystem.

FIG. 9 depicts an illustrative example of a distal subsystem.

FIG. 10 depicts an example of a proximal subsystem, including a controller (e.g., a proximal controller module) and a set of scaffolds.

FIG. 11 depicts an example of a set of scaffolds.

FIG. 12 depicts an example including a set of scaffolds and a tissue interface.

FIG. 13 depicts another example including a set of scaffolds and a tissue interface.

FIG. 14 depicts an example of an implanted system.

FIG. 15 depicts an image of an example of a tissue interface, including cells seeded into a set of scaffolds.

### DETAILED DESCRIPTION

The following description of the embodiments of the invention is not intended to limit the invention to these embodiments, but rather to enable any person skilled in the art to make and use this invention.

### 1. Overview

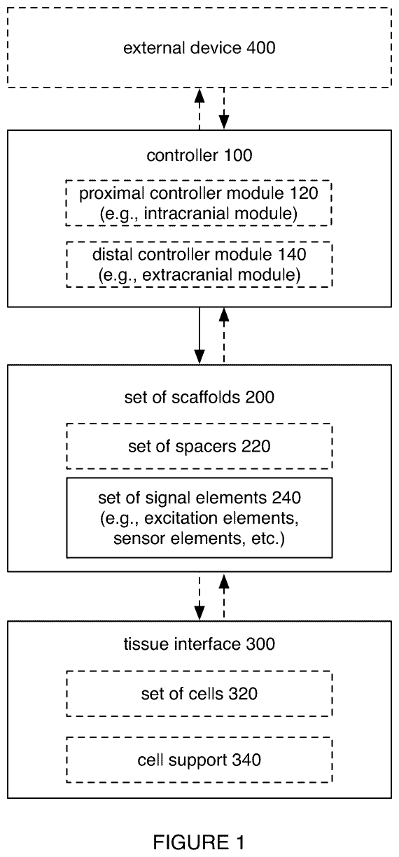

As shown in FIG. 1, the system 10 can include: a controller 100, a set of scaffolds 200, and a tissue interface 300. The system 10 can optionally include and/or be used with an external device 400. However, the system 10 can additionally or alternatively include any other suitable components.

In variants, the system 10 (e.g., an intracortical brain-computer interface) can function to transmit information to a user's brain and/or receive information from a user's brain. In specific examples, the system 10 can perform motor control, speech decoding, sensory restoration, sensory augmentation, a combination thereof, and/or other functions.

### 2. Examples

In an example, the system can include an intracortical brain-computer interface that can be embedded in-situ on the surface of the brain. In a specific example, the system can include an implant, wherein the implant includes: a controller, a set of stacked scaffolds, and a set of engineered cells seeded (e.g., in 3 dimensions) between adjacent pairs of scaffolds. For example, the set of stacked scaffolds can be dies arranged in parallel layers with spacers between adjacent pairs of dies. Each scaffold can include an integrated circuit and an array of signal elements, including excitation elements (e.g., μLEDs) that can excite the engineered cells and/or sensor elements (e.g., receiving electrodes) that can record signals (e.g., action potentials) from the engineered cells. In a specific example, each scaffold can include excitation elements on the left face of the scaffold (e.g., interfacing with a first group of engineered cells) and sensor elements on the right face of the scaffold (e.g., interfacing with a second group of engineered cells). In an example, the set of engineered cells can include optogenetically modified neurons (e.g., genetically modified to express transgenic proteins sensitive to the wavelength of light emitted by the μLEDs). The bodies of the engineered cells can optionally be retained in a hydrogel, wherein the engineered cells can project axons and/or dendrites through the hydrogel to interface with the native brain tissue. In an illustrative example, the excitation elements can emit light, wherein recipient engineered cells can stimulate the native brain tissue in response to detecting the light. In another illustrative example, the engineered cells can be stimulated by native brain tissue signals, wherein sensor elements can detect associated signals in the engineered cells in response to the stimulation.

### 3. Technical Advantages

Variants of the technology can confer one or more advantages over conventional technologies.

First, conventional intracortical brain-computer interfaces are hindered by a tradeoff between increasing bandwidth (e.g., channel count) and reducing damage to the brain; for conventional intracortical brain-computer interfaces, as bandwidth increases, the number of damaged brain cells also increases due to the increased implant size. Variants of the technology can provide a scalable intracortical brain-computer interface with a large number of channels (e.g., at least 1000, at least 5000, at least 10000, at least 50000, etc.) that can mitigate damage to the brain. In an example, the system includes a set of stacked scaffolds seeded in three dimensions with a high-density of engineered cells between the scaffolds. The engineered cells (e.g., engineered neurons) can grow projections to interface with the native cortex tissue, enabling the system to receive and/or send signals to the brain with minimal or no damage to the native neurons.

Second, variants of the technology can restore and/or augment one or more senses of a user, treat neurological diseases and/or disorders (e.g., amyotrophic lateral sclerosis, epilepsy, Alzheimer's disease, stroke, Parkinson's disease, motor impairments, etc.), perform motor control, perform speech decoding, and/or otherwise interface with a user's brain. In an example, the system can both record and stimulate engineered cells interfacing with neurons in the brain (e.g., recording from the same cells that are being simulated). In an illustrative example, the system can receive information associated with brain dynamics (e.g., detecting a seizure), and emit signals to the brain based on the received information (e.g., triggering stimulation to treat the seizure). In another illustrative example, an artificial intelligence model (e.g., reinforcement learning model) can be integrated into the brain via the intracortical brain-computer interface (e.g., the model can ingest signals received from the brain via the implant, and can send model outputs to the brain via the implant).

Third, conventional methods of interfacing with neurons require large capacitive electrode systems with cumbersome hermetic feedthroughs (to seal implanted electrodes). In variants, this technology can provide content to the brain (e.g., via RGCs, via neurons, etc.) using a display implant that has a higher resolution (e.g., a 1:1 mapping of cells to light emission systems on the display), a higher number of signal elements, and/or a smaller form factor with fewer or no hermetic feedthroughs. In an example, variants of the technology can be scalable (e.g., to thousands of pixels, to hundreds of thousands of pixels, etc.).

However, further advantages can be provided by the system and method disclosed herein.

### 4. System

As shown in FIG. 1, the system 10 can include: a controller 100, a set of scaffolds 200, and a tissue interface 300. The system 10 can optionally include and/or be used with one or more of: an external device 400, a computing system (e.g., local, remote, distributed, etc.), a database (e.g., a system database, a third-party database, etc.), user interface, and/or any other components. The computing system can include one or more: CPUs, GPUs, TPUs, custom FPGA/ASICS, microprocessors, servers, cloud computing, and/or any other suitable components. The computing system can be local, remote (e.g., cloud computing server, etc.), distributed, and/or otherwise arranged relative to any other system or module. The user interface can receive one or more inputs (e.g., from a user), display one or more outputs (e.g., processed and/or unprocessed data received from the sensor elements, model outputs, etc.), display any other parameters, and/or otherwise function.

The system 10 can be used with a tissue of interest of a user. For example, the system 10 can interface with the tissue of interest. In a specific example, the system 10 can be physically coupled to the tissue of interest (e.g., via tissue anchors). In another specific example, the system 10 can send signals to and/or receive signals from the tissue of interest (e.g., via the tissue interface). The tissue of interest preferably includes a brain (e.g., brain surface, cerebrum, cortex, etc.), but can alternatively include a brain stem, eye, spinal cord, ear, muscle, skin, nerve, and/or any other body region of the user. The user can be a human, an animal (e.g., rabbit, chimpanzee, mouse, rat, etc.), and/or any other organism. The system 10 and/or components therein are preferably implanted at one or more implantation locations (e.g., locations in, on, and/or near the tissue of interest). Examples of implantation locations include locations within or on the surface of: the brain, dura mater, pia mater, cortex, skull, skin, muscle, and/or any other implantation location.

As used herein, the z-axis is defined as extending away from the tissue of interest (e.g., away from the brain surface and towards the skull). As used herein, the x-axis is defined as orthogonal to a face of a scaffold (e.g., towards an adjacent scaffold). In an example, the height of a scaffold is along the z-axis, the thickness of a scaffold is along the x-axis, and the width of a scaffold is along the y-axis. The coordinates, as used herein, are intended only as a reference and are not intended to restrict the orientation of the system 10 relative to a global coordinate system. The system 10 can be arranged in any orientation (e.g., where the x-axis corresponds to a vertical axis, where the y-axis corresponds to a vertical axis, where the z-axis corresponds to a vertical orientation, etc.).

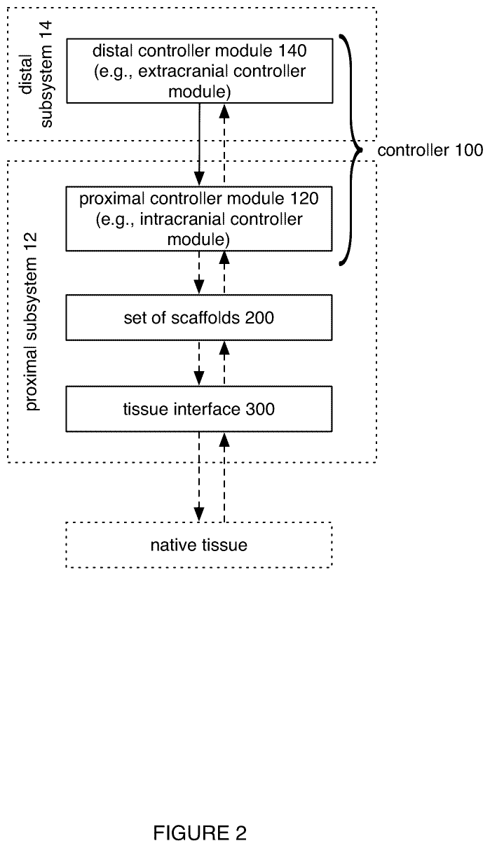

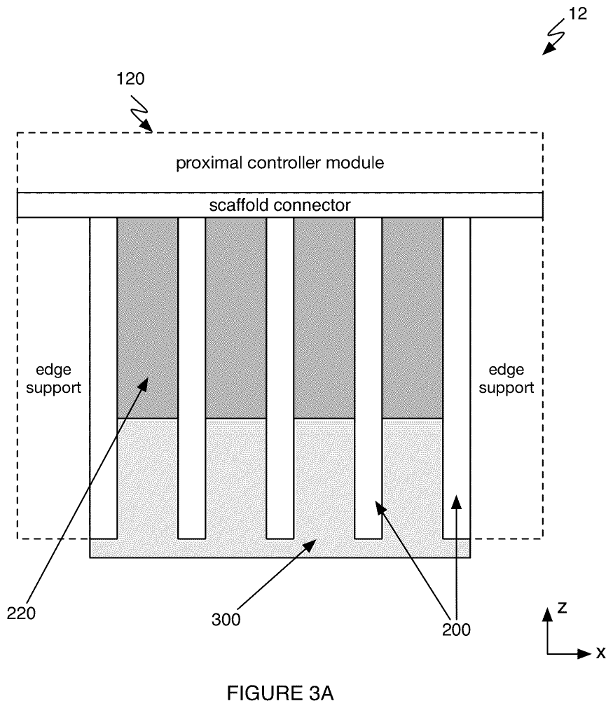

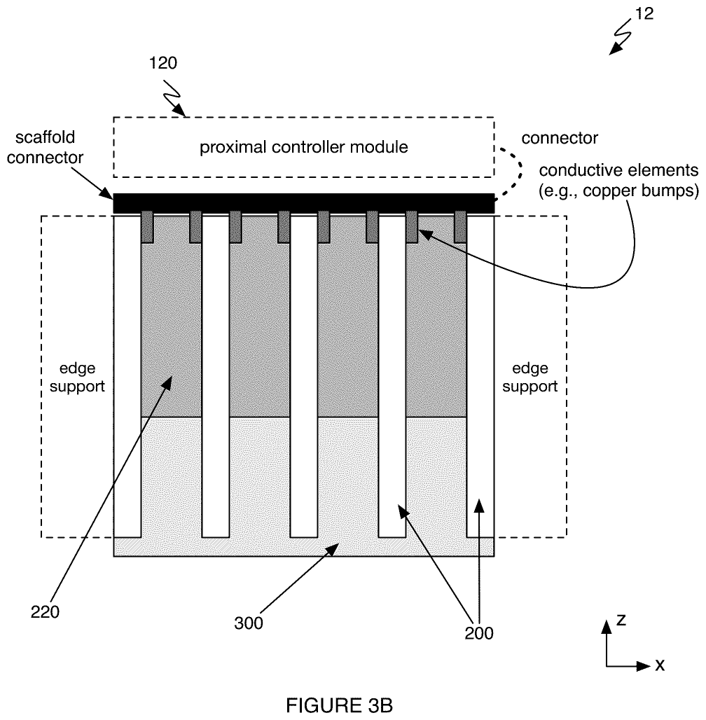

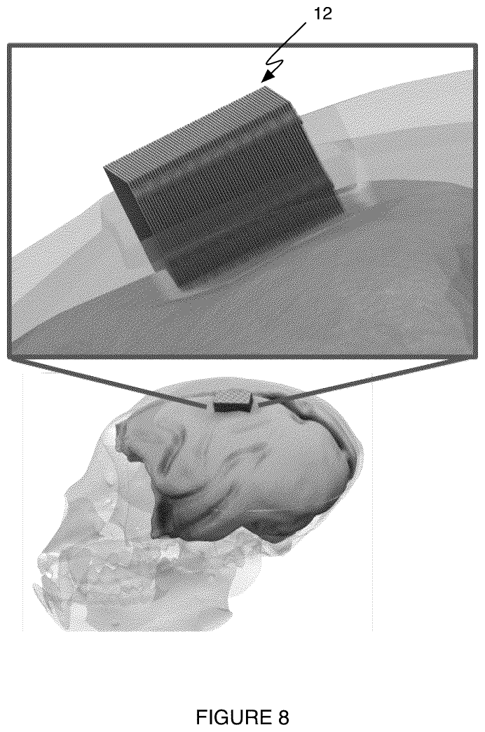

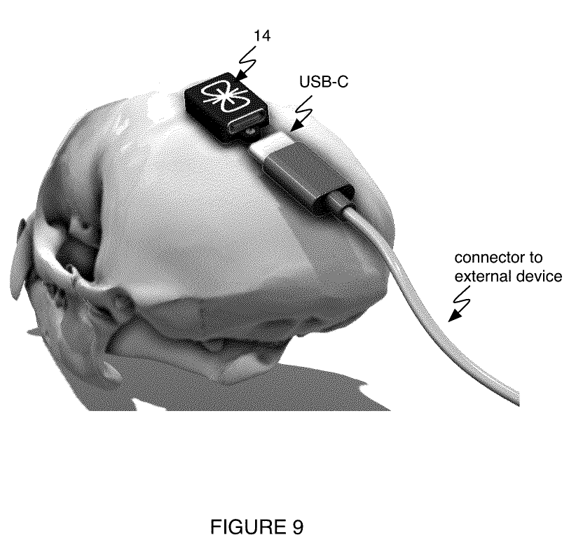

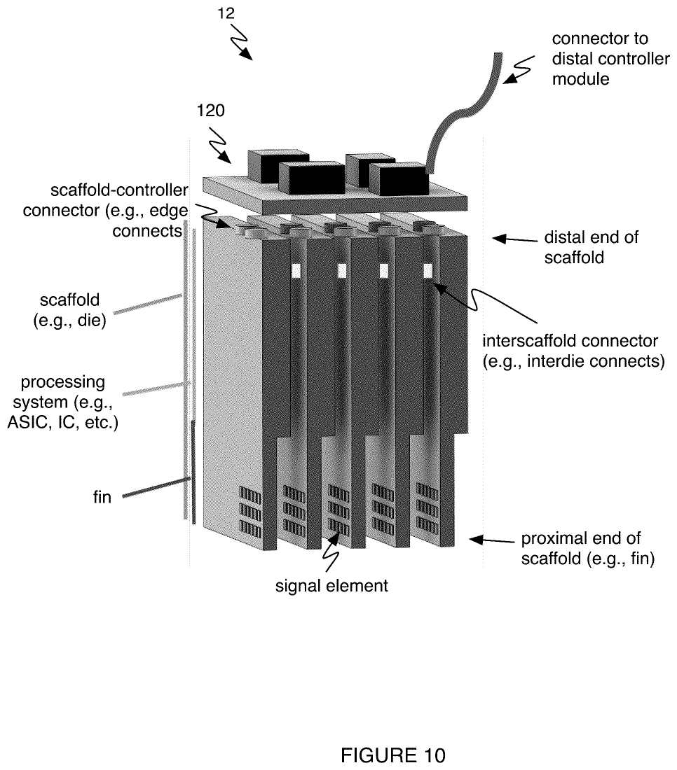

The system 10 can optionally include multiple subsystems. An example is shown in FIG. 2. For example, the system 10 can include a proximal subsystem 12 implanted proximal to the tissue of interest (e.g., in contact with the tissue of interest) and a distal subsystem 14 implanted distal to the tissue of interest (e.g., at or near the skin surface, crossing the skin surface, crossing the skull, etc.). In an example, the proximal subsystem 12 (e.g., intracranial subsystem) can include a proximal controller module 120 (e.g., intracranial controller module), the set of scaffolds 200, the tissue interface 300, and/or any other system components. Examples are shown in FIG. 3A, FIG. 3B, and FIG. 10. In an example, the distal subsystem 14 (e.g., an extracranial subsystem) can include a distal controller module 140 (e.g., extracranial controller module) of the controller 100 and/or any other system components. In an example, the extracranial subsystem can be implanted partially or completely external to the skull (e.g., at or near the skin surface, passing through the skin surface, etc.). An example is shown in FIG. 9. In a first specific example, the extracranial subsystem can cross the skin surface (e.g., the extracranial subsystem is exposed outside the skin). In a second specific example, the extracranial subsystem can be implanted beneath the skin surface (e.g., where the skin is sutured to cover the extracranial subsystem). In an example, the intracranial subsystem can be implanted partially or completely internal to the skull. An example is shown in FIG. 8. In a first specific example, a section of the skull is removed, and the intracranial subsystem extends from the surface of the brain to a location (e.g., height) within (e.g., at or below) the pre-removal location of the inner surface of the section of the skull. In an illustrative example, the intracranial subsystem is located within between the brain and the inner surface of the skull (e.g., beneath the skull). In a second specific example, a section of the skull is removed, and the intracranial subsystem extends from the surface of the brain to a location (e.g., height) outside (e.g., above) the pre-removal location of the inner surface of the section of the skull. In an illustrative example, the intracranial subsystem passes partially or completely through the skull. However, subsystems can be otherwise configured.

The system 10 can optionally include a housing around all or a portion of the system 10 and/or components therein (e.g., the proximal subsystem 12 and/or the distal subsystem 14). The housing can be: a coating (e.g., an encapsulant), a wrapping, a jacket, a cap, and/or any other material covering all or a portion of the system 10 and/or components therein. Examples of housing materials can include: silicone, polyether-ether-ketone (PEEK), metals (e.g., titanium), a combination thereof, and/or any other suitable material. The housing(s) can function to seal system components, provide flanges for anchoring, facilitate implantation, and/or perform other functions. In a first specific example, the proximal subsystem 12 can include a housing (e.g., a silicon jacket) that includes one or more flanges, wherein the flange(s) are used as anchoring points for one or more tissue anchors (e.g., brain anchors). In a second specific example, the distal subsystem 14 can include a housing that includes one or more flanges, wherein the flange(s) are used as anchoring points for one or more skull anchors. In another illustrative example, the housing for the distal subsystem 14 can include a cap exposed beyond the skull. The cap can be polyether-ether-ketone (PEEK), metal (e.g., titanium), and/or any other suitable material.

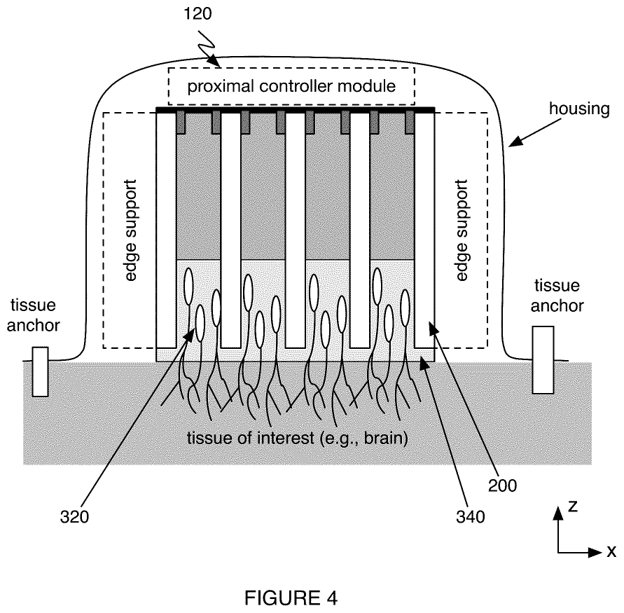

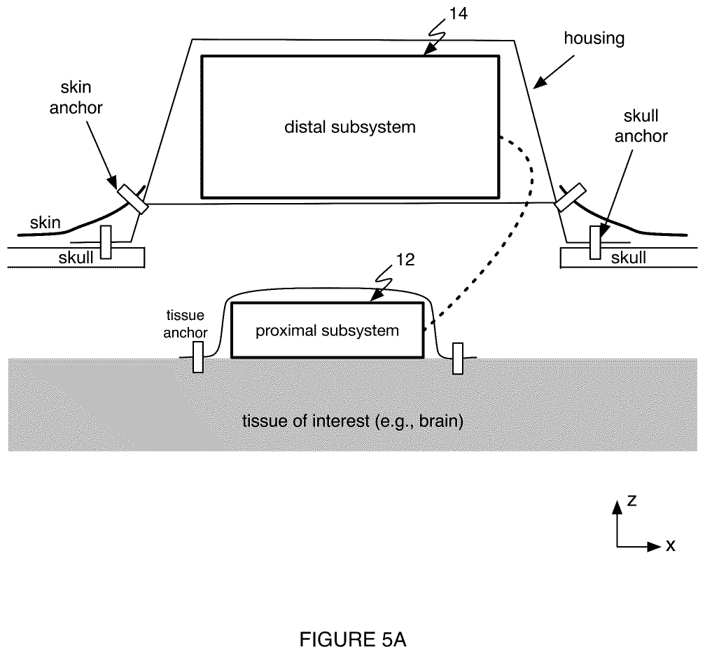

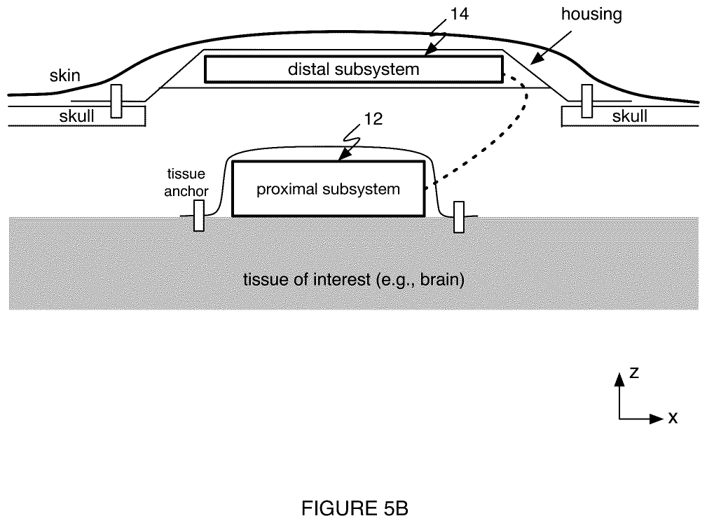

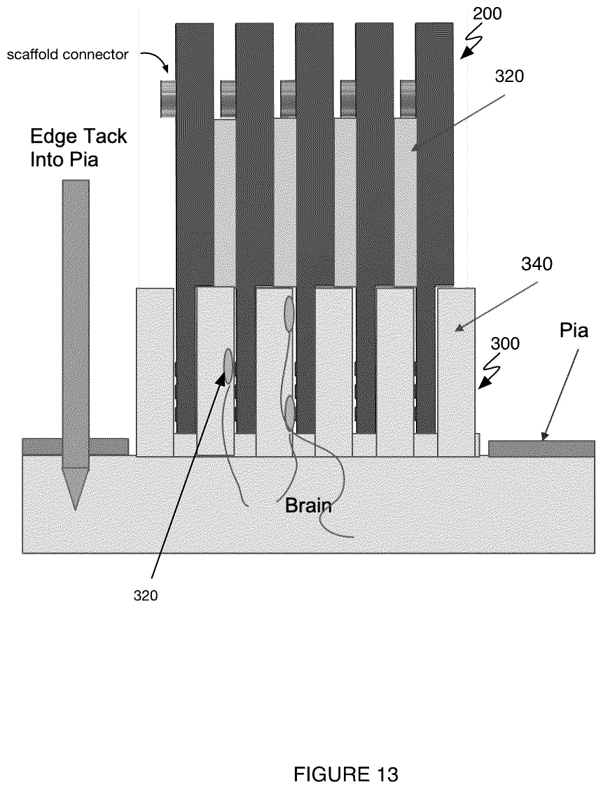

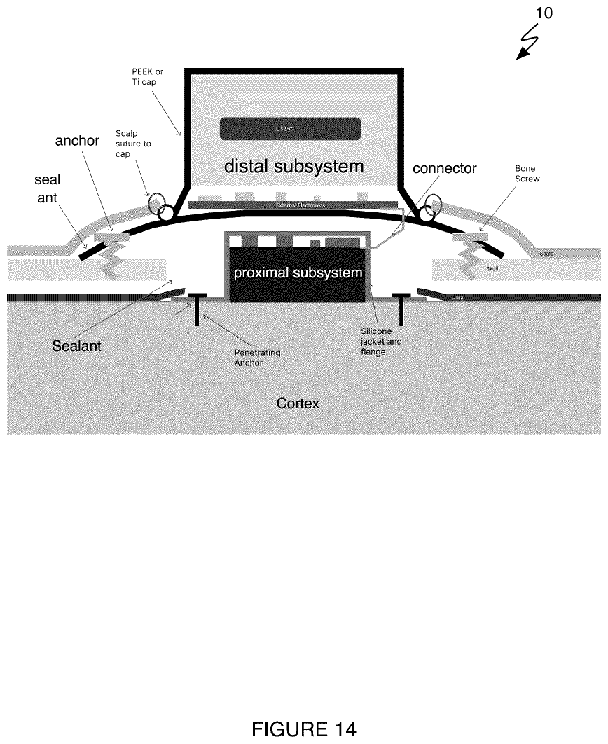

The system 10 and/or components therein can optionally be anchored (e.g., adhered, secured, etc.) to tissue (e.g., skin, bone, dura mater, pia mater, etc.) at an implantation location. Anchoring methods can include: tissue adhesives, extrusions that act as anchors, packing, anchors (e.g., tacks, pins, screws, etc.) inserted through the component (e.g., through a flange in a housing around the system 10 and/or components therein), inducing fibrotic growth in or around the system 10, anchoring via the tissue interface 300, a combination thereof, and/or any other method. Examples are shown in FIG. 4, FIG. 5A, FIG. 5B, FIG. 13, and FIG. 14. In an illustrative example, one or more anchors can secure the proximal subsystem 12 (e.g., intracranial subsystem) to the brain tissue. In a specific example, the system 10 can include an anchor configured to secure the set of scaffolds 200 to the surface of the brain of a user. In another illustrative example, one or more bone screws can secure the distal subsystem 14 (e.g., extracranial subsystem) to the skull. In another illustrative example, sutures can secure the distal subsystem 14 to skin. The system 10 and/or components therein can optionally be sealed to tissue at the implantation location(s). Examples of sealants can include dental acrylic, silicone adhesive, and/or any other sealants.

The system 10 can optionally include a spring, which can function to bias the proximal subsystem 12 and/or other system components against the tissue of interest and/or against other tissue at an implantation location. For example, the spring can be connected to the intracranial subsystem, pressing the intracranial subsystem against the brain surface (e.g., pia mater, cortex tissue, etc.). The spring can be positioned between the proximal subsystem 12 and the distal subsystem 14, between the proximal subsystem 12 and other tissue (e.g., skull, skin, dura mater, etc.), and/or any other components. The spring can be a coil spring, a plate (e.g., cambered plate), deformable material, and/or any other spring. The spring material can include metal, plastic, a hydrogel, an elastomeric material, and/or any other material.

The controller 100 functions to control the set of signal elements 240 (e.g., excitation elements, sensor elements, etc.) and/or other electronic components of the set of scaffolds 200. For example, the controller 100 can transmit control instructions (e.g., excitation parameters, sensor parameters, etc.) to the set of scaffolds 200 and/or receive data from the set of scaffolds 200. The controller 100 can additionally or alternatively function to: transmit and/or receive information from an external device 400; process data (e.g., compress data, convert data, transform data, etc.); perform safety checks; provide power to one or more system components (e.g., to the set of scaffolds 200); regulate power; acquire data; and/or perform other functions. In a specific example, the controller can provide power to the set of scaffolds 200, wherein the controller 100 itself can be powered by an external power source and/or an onboard power source. The controller 100 and/or components therein are preferably physically connected to the set of scaffolds 200, but can alternatively be not physically connected to the set of scaffolds 200.

In examples, the controller 100 can include one or more: power components (e.g., power source, power receiver components, power rectification and/or regulation components, etc.), communication elements, processing systems (e.g., processor, memory, data processing circuitry, logic, microcontroller, integrated circuits, etc.), filters (e.g., capacitors, resistors, etc.), controller sensors, analog-to-digital converter (ADC), ultrasonic backscatter reduction components, optical backscatter reduction components, and/or any other system component. Integrated circuits can include: field-programmable gate array (FPGA), system on a chip, application-specific integrated circuit (ASICs), and/or any other integrated circuit. Communication elements can include: receivers, transmitters, transceivers, antenna, wired connections, and/or any other components. Communication elements can be wired and/or wireless (e.g., radio frequency (RF), infrared (IR), Bluetooth, BLE, NFC, etc.).

The controller sensors can optionally function to measure a controller state, a user state, tissue state, and/or collect any other data. Examples of controller sensors can include moisture sensor (e.g., humidity sensor), temperature sensor, pose and/or motion sensor (e.g., IMU), metabolism sensor, pH sensor, current sensor, voltage sensor, ADC (e.g., to measure power), and/or any other sensor. The controller 100, external device 400, the set of signal elements 240, and/or any other components can optionally be controlled based on data received from the controller sensors. In a first example, power can be halted to one or more components in response to detection or occurrence of a predetermined controller state (e.g., indicating water ingress, temperature above a threshold, electrical shorts, etc.). In a second example, data from the set of signal elements 240 can be processed (e.g., reducing noise) based on the sensor data.

The controller 100 can optionally include one or more modules. For example, the controller 100 can include a proximal controller module 120 proximal to the tissue of interest and/or a distal controller module 120 distal to the tissue of interest. The proximal controller module 120 can function to interface with the set of scaffolds 200 and/or perform other controller 100 functions. The distal controller module 140 can function to process data, provide power, interface with an external device 400 (e.g., wirelessly, via a wired connection such as a USB-C, etc.), and/or perform other controller 100 functions. In an example, the distal controller module 140 can include: power components (e.g., power source, power receiver, regulators, etc.), filters, processing systems (e.g., processor, memory, data processing circuitry, logic, microcontroller, integrated circuits, etc.), controller sensors, communication elements, and/or any other controller 100 components. In an example, the proximal controller module 120 can include: power components (e.g., power receiver, regulators to filter noise and/or for other functions, etc.), filters, processing systems, controller sensors, communication elements, and/or any other controller 100 components.

In a first variant, the controller 100 can include a proximal controller module 120 (e.g., intracranial controller module) and a distal controller module 140 (e.g., extracranial controller module). For example, the distal controller module 140 can be connected to the proximal controller module 120 (e.g., via a wired connection) and optionally the external device 400; the proximal controller module 120 can be connected to the set of scaffolds 200. In specific examples, the proximal controller module 120 can be connected to the set of scaffolds 200 via: a direct bond to the scaffolds, a direct bond to a scaffold connector, a wired connection to the scaffolds, a wired connection to a scaffold connector, and/or otherwise connected. In a specific example, the distal controller module 140 can be connected to the external device 400 via a wired and/or a wireless connection. The proximal controller module 120 and the distal controller module 140 are preferably physically connected (e.g., by a cable and/or other wired connection), but can alternatively be wirelessly connected. In a specific example, the connection between the proximal controller module 120 and the distal controller module 140 can be a flexible connector. In a specific example, a wired connection between the proximal controller module 120 and the distal controller module 140 can include one or more bends in the wired connection (e.g., for strain relief, to facilitate implantation, etc.). In a specific example, the controller 100 can include a proximal controller module 120 and a distal controller module 140, wherein the proximal controller module 120 is coupled to the set of scaffolds 200, wherein the distal controller module 140 is anchored to the skull of the user, and wherein the distal controller module 140 is communicatively coupled to an external device 400.

In a second variant, the controller 100 can include (only) a distal controller module 140 (e.g., extracranial controller module). For example, the distal controller module 140 can be connected to the set of scaffolds 200 and optionally the external device 400. In specific examples, the distal controller module 140 can be connected to the set of scaffolds 200 via: a wired connection to the scaffolds, a wired connection to a scaffold connector, and/or otherwise connected. In a specific example, the distal controller module 140 can be connected to the external device 400 via a wired and/or a wireless connection.

In a third variant, the controller 100 can include (only) a proximal controller module 120 (e.g., intracranial controller module). For example, the proximal controller module 120 can be connected to the set of scaffolds 200 and an external device 400. In specific examples, the proximal controller module 120 can be connected to the set of scaffolds 200 via: a direct bond to the scaffolds, a direct bond to a scaffold connector, a wired connection to the scaffolds, a wired connection to a scaffold connector, and/or otherwise connected. In a specific example, the proximal controller module 120 can be connected to the external device 400 via a wireless connection.

However, the controller 100 can be otherwise configured.

The set of scaffolds 200 functions to emit excitation signals and/or to detect signals from cells within the tissue interface 300. The set of scaffolds 200 can be connected to the controller 100 (e.g., to the proximal controller module 120), to the tissue interface 300, and/or to any other system component. For example, the set of scaffolds 200 can receive control instructions (e.g., excitation parameters, sensor parameters, etc.) from the controller 100, and can emit excitation signals (via excitation elements) and/or receive cell signals (via sensor elements) according to the control instructions. For example, the set of scaffolds 200 can receive control instructions (e.g., excitation parameters, sensor parameters, etc.) from the controller 100, and can emit excitation signals (via excitation elements) and/or receive cell signals (via sensor elements) according to the control instructions.

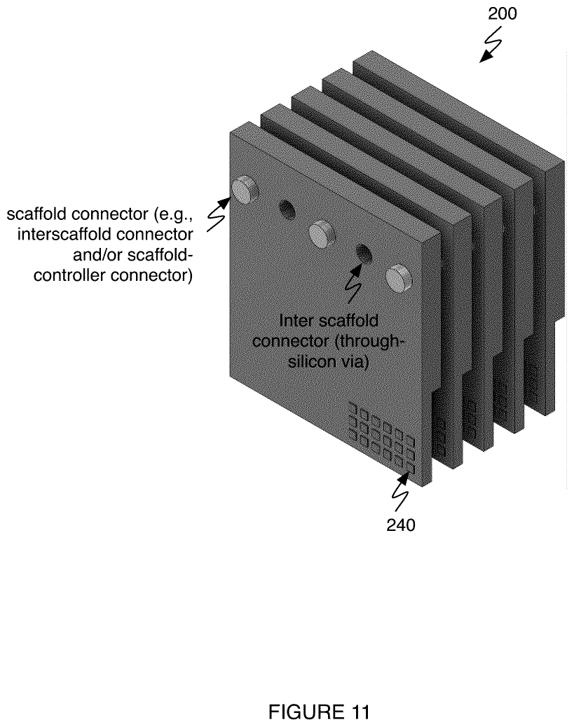

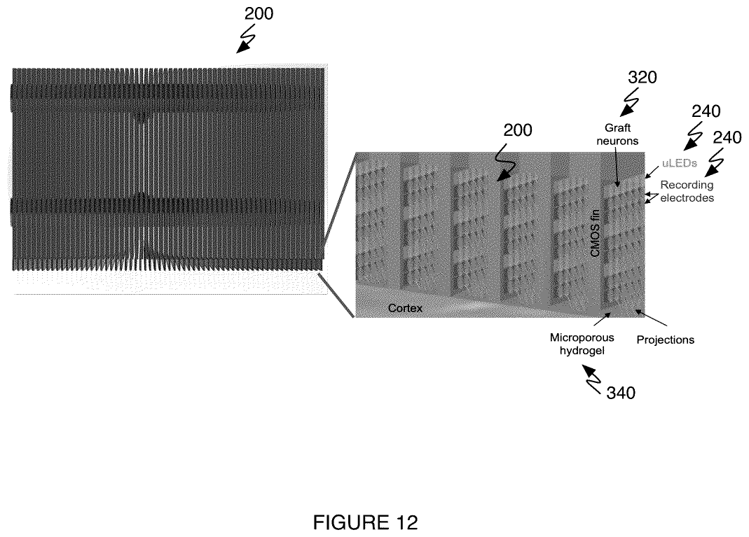

Each scaffold can include one or more of: signal elements (e.g., one or more arrays of signal elements), drivers (e.g., μLED drivers), power components (e.g., internal regulation), processing systems (e.g., integrated circuits (ICs)), registers, multiplexers, ADCs, and/or any other system component. Examples are shown in FIG. 11 and FIG. 12. Scaffolds preferably include a silicon backing, but can additionally or alternatively include any other material. In an example, each scaffold can include one or more ICs (e.g., ASICs). In a specific example, a scaffold can include a die containing an array of signal elements (e.g., an array of excitation elements and/or an array of sensor elements) and an IC (e.g., including drivers for excitation elements, internal regulation, registers, multiplexers, an ADC, etc.). In an illustrative example, each scaffold can be configured with the set of signal elements 240 on a proximal end (e.g., fin) of the scaffold configured to be proximal to the tissue of interest, and the IC on a distal end of the scaffold configured to be distal to the tissue of interest (e.g., and proximal to the controller 100). Scaffolds in the set of scaffolds 200 can contain the same or different signal elements, the same or different signal element configurations (e.g., location of the signal elements on the scaffolds), the same or different IC components, and/or can be otherwise configured. In an illustrative example, a subset of the set of scaffolds 200 can include ADCs (e.g., where connections between the scaffolds enable a scaffold without an ADC to use an ADC on a neighboring scaffold). Scaffolds can optionally ingest and/or output data using serial protocols (e.g., using Inter-Integrated Circuit, Serial Peripheral Interface, etc.). In a specific example, signals output from each scaffold (e.g., data received from sensor elements; output via an ADC on the scaffold) can be serialized across the set of scaffolds 200 and transmitted to the controller 100 (e.g., to the proximal controller module 120).

In an example, the number of scaffolds in the set of scaffolds 200 can be between 1-10,000 or any range or value therebetween (e.g., 1-5, 5-10, 10-50, 50-100, 100-256, at least 2, at least 4, at least 8, at least 10, at least 64, at least 100, etc.). Each scaffold is preferably planar, but can alternatively be any other geometry. In an example, the thickness of a scaffold (e.g., in the x-direction) can be between 5 μm-1000 μm or any range or value therebetween (e.g., 20 μm-60 μm, 20 μm, 50 μm, less than 500 μm, less than 100 μm, less than 20 μm, etc.). In a first embodiment, the thickness is constant along the height of the scaffold (e.g., in the z-direction). In a second embodiment, the thickness can be variable. In an example, the thickness at the fin (proximal to the tissue of interest) can be less than the thickness at other locations on the scaffold. In another example, the thickness can taper at the edge of the fin (e.g., to facilitate cell loading).

The scaffolds are preferably stacked in parallel layers (e.g., at intervals along the x-axis), but can additionally or alternatively include non-parallel scaffolds (e.g., a second stack of scaffolds orthogonal to a first stack of scaffolds) and/or can be arranged in any other geometry. In an example, the footprint of the set of scaffolds 200 (e.g., on the tissue of interest; in the xy-plane) can be between 0.5 mm²-5 cm² or any range or value therebetween (e.g., 10 mm²-100 mm², 50 mm², etc.). In an example, the center-to-center distance between scaffolds (e.g., in the x-direction) can be between 30 μm-1000 μm or any range or value therebetween (e.g., 50 μm-100 μm, approximately 80 μm, less than 150 μm, at least 50 μm, at least 100 μm, at least 200 μm, etc.). In an example, the distance between adjacent fins (e.g., the gap between the surface of a first fin to the surface of the adjacent fin; the thickness of a spacer as described below; etc.) can be between 20 μm-1000 μm or any range or value therebetween (e.g., 20 μm-100 μm, 100 μm-1000 μm, approximately 500 μm, at least 100 μm, at least 200 μm, etc.). The end faces of the stack of scaffolds can optionally be adhered to an edge support. In an example, the thickness of the edge support can be between 10 μm-10,000 μm or any range or value therebetween. The material of the edge support can be glass, a polymer, metal (e.g., titanium), and/or any other suitable material.

A set of spacers 220 can optionally interface with the set of scaffolds 220. For example, a spacer can be positioned between each pair of adjacent scaffolds. The material of the set of spacers 220 can be glass, a polymer (e.g., liquid crystal polymer), metal (e.g., titanium), any encapsulation material, and/or any other suitable material. The set of spacers 220 can optionally extend partially down the set of scaffolds 200 (e.g., in the z-direction; from the distal end of a scaffold towards the fin), forming an open trench between fins. In an example, the height of a spacer (e.g., in the z-direction) can be between 50 μm-5000 μm or any range or value therebetween (e.g., 200 μm-400 μm). In an example, the height of a spacer (e.g., in the z-direction) can be between 10%-90% of the height of the scaffolds or any range or value therebetween (e.g., at least 25%, at least 50%, etc.). In an example, the thickness of a spacer (e.g., thickness of the trench) can be between 20 μm-1000 μm or any range or value therebetween (e.g., 20 μm-100 μm, 100 μm-1000 μm, approximately 500 μm, at least 100 μm, at least 200 μm, etc.). In an example, the depth of the trench between fins (e.g., in the z-direction) can be between 50 μm-5000 μm or any range or value therebetween (e.g., 200 μm-400 μm).

Scaffolds (e.g., fins) can optionally be coated with proteins (e.g., growth factors, brain proteins, etc.), poly-d-lysine, laminin, and/or other materials. Scaffolds (e.g., fins) can optionally undergo one or more surface modifications. For example, the surface of the scaffolds (e.g., the surface of the fins) can undergo plasma activation. In variants, this can increase the hydrophilicity of the surface of the scaffold, increasing adhesion between the scaffolds and the cell support 340.

The set of scaffolds 200 can optionally include one or more scaffold connectors. Scaffold connectors can include: interscaffold connectors (e.g., interdie connectors) connecting multiple scaffolds, scaffold-controller connectors connecting one or more scaffolds to the controller 100 (e.g., to the proximal controller module 120), and/or any other connectors. In an example, scaffold connectors can include connections through scaffolds (e.g., through-silicon via) and/or edge out connectors (e.g., connections along the distal edge of the scaffolds). The scaffold connectors can optionally include an interposer layer between the controller 100 and the set of scaffolds 200.

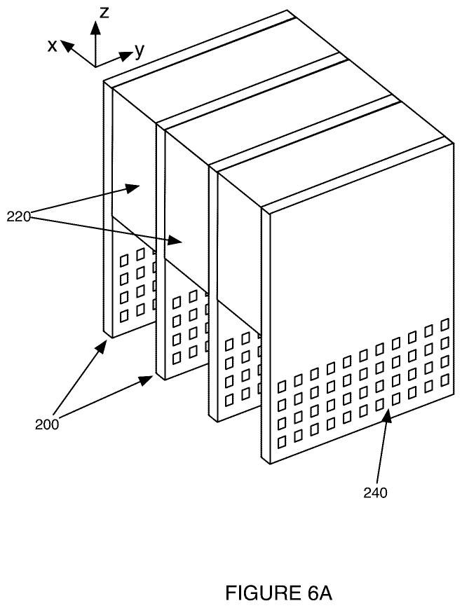

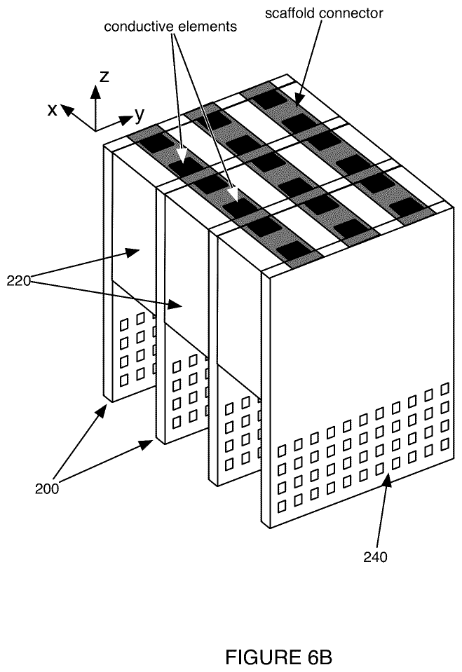

The set of spacers 220 can optionally include a set of raised conductive elements (e.g., copper bumps), wherein the scaffold connector connects multiple raised conductive elements. Examples are shown in FIG. 3B and FIG. 6B. For example, a spacer between a left scaffold and a right scaffold can include a first raised conductive element in contact with the right face of the left scaffold and second raised conductive element in contact with the left face of the right scaffold; the scaffold connector can then connect the first raised conductive element to the second raised conductive element (e.g., thus connecting the left scaffold to the right scaffold). In a specific example, the face of the distal edge of each scaffold is ground down to reveal conductive elements in the scaffolds, enabling electrical connectivity between the raised conductive elements on the set of spacers 220 and the set of scaffolds 200. In an illustrative example, the scaffold connector can be a metal trace along the top edge of the set of scaffolds 200, connecting all scaffolds in the set of scaffolds 200 via the raised conductive elements. In an illustrative example, the scaffold connector can be bonded (e.g., wire bonded) to a cable connected to the controller 100 (e.g., to the proximal controller module 120 and/or to the distal controller module 140).

In an illustrative example, manufacturing the set of scaffolds 200 can include one or more of: forming individual scaffolds (e.g., thinning a full thickness silicon wafer), stacking the scaffolds with a spacer (e.g., liquid crystal polymer) between each pair of scaffolds, compressing the scaffolds, bonding the scaffolds (e.g., polymer bonding, oxide-oxide bonding, etc.; optionally including etching holes within the scaffolds to facilitate the bonding), grinding the face of the distal edge of each scaffold is to reveal conductive elements in the scaffolds, connecting the scaffolds to the controller 100 (e.g., to the proximal controller module 120), and/or any other suitable steps.

The set of signal elements 240 can include excitation elements, sensor elements, and/or any other element. Each signal element in set of signal elements 240 can interface with (e.g., record from and/or excite) 1 cell, more than 1 cell (e.g., at least 2, at least 3, etc.), 0 cells, a variable number of cells, a randomly determined number of cells, all cells within a trench, all cells within a trench that are less than a predetermined distance from the signal element (e.g., 100 μm, 50 μm, 10 μm, 5 μm, etc.), and/or any other number of cells. In an illustrative example, a cell in the tissue interface 300 can be stimulated via an excitation element on a scaffold, and a sensor element (on the same scaffold or an adjacent scaffold) can record signals from the same cell.

In an example, a scaffold face can include between 10-50,000 signal elements or any range or value therebetween (e.g., 1,000-10,000; 2,000-5,000; at least 10; at least 100; at least 1,000; etc.). In a specific example, a scaffold face can include between 10-100,000 sensor elements (e.g., recording electrodes) or any range or value therebetween (e.g., 1,000-10,000; 2,000-5,000; at least 10; at least 100; at least 1,000; etc.). In another specific example, a scaffold face can include between 10-100,000 excitation elements (e.g., μLEDs) or any range or value therebetween (e.g., 1,000-10,000; 2,000-5,000; at least 10; at least 100; at least 1,000; etc.). In an example, the total number of signal elements across the set of scaffolds 200 can be between 1000-100million or any range or value therebetween (e.g., at least 50k, at least 100k, at least 395k, at least 1 million, etc.). In an example, the diameter of a signal element can be between 1 μm-1 mm or any other range or value therebetween. In an example, the pitch in an array of signal elements on a scaffold (e.g., center-to-center distance between signal elements in the set of signal elements 240) can be between 5 μm-10 mm or any range or value therebetween.

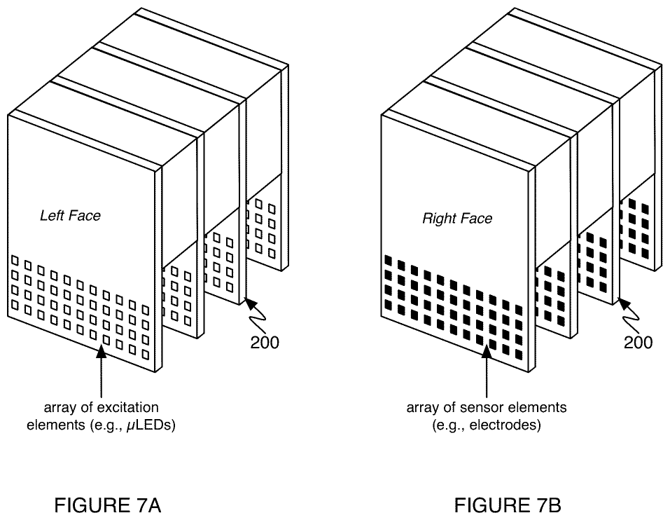

Signal elements can be located on a single face of a scaffold or multiple faces of a scaffold (e.g., opposing faces of a scaffold). Examples are shown in FIG. 6A, FIG. 6B, FIG. 7A, and FIG. 7B. In an example the set of signal elements 240 can include one or more arrays of signal elements on each scaffold of the set of scaffolds 200. In a specific example, the set of signal elements 240 includes one array of signal elements on the two end scaffolds (e.g., the two scaffolds flanking the set of scaffolds 200) and two arrays of signal elements on the intervening scaffolds (e.g., all scaffolds between the two end scaffolds). In a second specific example, the set of signal elements 240 includes two arrays of signal elements on all scaffolds in the set of scaffolds (e.g., where the array of signal elements on the outer face of each end scaffold is not used). In a specific example, sensor elements (e.g., an array of recording electrodes) and excitation elements (e.g., an array of μLEDs) are located on opposing faces of a scaffold. Examples are shown in FIG. 7A and FIG. 7B. In an illustrative example, for a trench defined by a left scaffold and a right scaffold, excitation elements are located on the right face of the left scaffold (forming the left side of the trench) and sensor elements are located on the left face of the right scaffold (forming the right side of the trench). In another illustrative example, for a trench defined by a left scaffold and a right scaffold, sensor elements are located on the right face of the left scaffold (forming the left side of the trench) and excitation elements are located on the left face of the right scaffold (forming the right side of the trench).

In an example, the system 10 can include: a first scaffold including an array of μLEDs on a first face of the first scaffold; a second scaffold including an array of recording electrodes on a first face of the second scaffold (e.g., where the first face of the first scaffold faces the first face of the second scaffold); a spacer separating the second scaffold from the first scaffold, the spacer positioned between the first face of the first scaffold and the first face of the second scaffold (e.g., where the spacer is in contact with the first face of the first scaffold and the first face of the second scaffold); a cell support (e.g., gel) positioned between the array of μLEDs and the array of recording electrodes; and a set of cells 320 (e.g., genetically modified cells) retained within the cell support, wherein the array of recording electrodes is configured to receive signals from the set of cells 320, wherein the array of μLEDs is configured to transmit light signals to the set of cells 320. In a specific example, the first scaffold further includes a second array of recording electrodes on a second face of the first scaffold, wherein the second face of the first scaffold is opposite the first face of the first scaffold, and wherein the second scaffold further includes a second array of μLEDs on a second face of the second scaffold, wherein the second face of the second scaffold is opposite the first face of the second scaffold.

A sensor element can include a light sensor system (e.g., detecting a wavelength of light emitted by cells in the tissue interface 300), an electrical sensor system (e.g., a recording electrode sensing a current signal and/or voltage signal from cells in the tissue interface 300), and/or any other signaling system. In a specific example, the sensor elements can be or include an array of recording electrodes. Sensor elements can measure signals (e.g., action potentials) from cells in the tissue interface 300. In specific examples, the sensor elements can measure the presence and/or concentration of one or more cellular molecules (e.g., calcium ions), cell response to an excitation signal, cell response to a signal from the tissue of interest, and/or any other cell response. However, sensor elements can be otherwise configured.

An excitation element can include a light emission system, an electrical emission system (e.g., emitting a current signal and/or voltage signal), and/or any other signaling system. The light emission system preferably includes a μLED, but can additionally or alternatively include laser diodes, phosphors, and/or any other light emission system. In a first specific example, the excitation elements can be or include an array of electrodes. In a second specific example, the excitation elements can be or include an array of μLEDs. The μLEDs (e.g., an array of μLEDs, multiple subarrays of μLEDs, etc.) can optionally be configured with a common cathode integration or a common anode configuration. The light parameters of the light emission system (e.g., wavelength, frequency, photon energy, intensity, flux, amplitude, etc.) preferably correspond to an optogenetic actuator (e.g., an opsin) associated with cells in the tissue interface 300, but can alternatively not be associated with an optogenetic actuator. In an example, the wavelength (e.g., spectral peak) can be between 400 nm-800 nm or any range or value therebetween.

The excitation elements can operate according to excitation parameters (e.g., excitation instructions) received from the controller 100. Excitation parameters can include spatial parameters (e.g., which excitation elements to operate), temporal parameters (e.g., when to initiate signal emission, length of signal emission, etc.), intensity and/or amplitude parameters (e.g., intensity and/or amplitude of light), light wavelengths, and/or any other parameters defining the signals emitted by the excitation elements. For example, the emission parameters can prescribe a timeseries of light array patterns (e.g., including wavelength, intensity, spatial information, state change instructions, and/or any other parameters for each excitation element), wherein each light array pattern encodes information (e.g., a content frame). The excitation parameters are preferably determined based on content (e.g., visual data, sensory data, other external information, artificial data, any other data, etc.), but can additionally or alternatively be determined based on cell state, signal element information (e.g., the current state of each signal element in the set of signal elements 240), controller state information, calibration information, and/or any other information. For example, the emission parameters can be determined such that the resulting excitation signals collectively encode the content.

The set of scaffolds 200 can optionally include and/or be coupled to one or more optics components (e.g., microoptical components). The optics components can function to collimate light, homogenize light, focus light, reduce light backscatter, and/or otherwise modify light emitting to or from the excitation elements. Examples of optics components can include: lenses (e.g., microlens, diffractive lens, metalens, etc.), back reflectors, a modified μLED shape, waveguides, a spacing gap, a combination thereof, and/or any other optics components.

However, the set of scaffolds 200 can be otherwise configured.

The tissue interface 300 functions to convert the excitation signals from the excitation elements to neural signals that can be interpreted by the brain and/or to produce action potentials (e.g., associated with neural signals received from the brain) that can be detected by the sensor elements.

The tissue interface 300 can include a set of cells 320, a cell support 340, and/or any other suitable components. All or a portion of the tissue interface 300 (e.g., the cell support 340 of the tissue interface 300) is preferably in contact with the tissue of interest, but can alternatively be separated from the tissue of interest (e.g., separated by pia mater), and/or otherwise positioned. In an example, the set of cells 320 can interface (e.g., directly or indirectly) with the tissue of interest. In a specific example, the set of cells 320 can interface with native neurons in the brain after growing projections (e.g., axons, dinitrides, etc.) into the brain tissue. For example, the set of cells 320 (e.g., genetically modified cells) can include axons and/or dendrites that extend out of the cell support (e.g., gel), wherein the axons and/or dendrites interface with native neurons in the brain of the user. In an illustrative example, when activated by excitation signals from the excitation elements, the set of cells 320 can evoke activity in the brain. In another illustrative example, when activated by neural signals from the native brain tissue, the set of cells 320 can produce action potentials that can be detected by sensor elements.

The set of cells 320 preferably includes neurons, but can additionally or alternatively include stem cells, neural progenitor cells, and/or any other cell type. In an example, the set of cells 320 can include cells derived from pluripotent stem cells. In a specific example, the set of cells 320 can include neurons (e.g., neurons derived from pluripotent stem cells) and/or neural progenitor cells (e.g., neural progenitor cells derived from pluripotent stem cells). The set of cells 320 can be cells derived from the user or cells derived from another organism. The set of cells 320 can include genetically modified cells, unmodified cells, and/or a combination thereof. Genetically modified cells can include optogenetically modified cells with light-sensitive biochemical signaling pathways (e.g., such that the cells produce biochemical signals in response to receiving certain wavelengths of light), cells modified to overexpress collagenous (e.g., to enable the cells to penetrating residual pia mater), hypoimmune cells (e.g., genetically modified to reduce immune response due to implantation), cells modified to include a small molecule killswitch (e.g., transfected with a killswitch gene), cells modified to include inducible transcription factors to drive cell fate, cells modified to include a calcium sensor, cells modified to contain engineered cell-surface molecules (e.g., to enable the cells to form specific synaptic connections), a combination thereof, and/or other genetically modified cells. Genetic modifications can optionally be inducible. Examples of cells that can be genetically modified include: organoids, cells selected from an organoid (e.g., a specific cell type), neurons, neural progenitor cells, stem cells (e.g., wherein the stem cells are genetically modified prior to differentiation), any animal cell (e.g., human cell), and/or any other cell. In a specific example, the genetically modified cells can include genetically modified neurons (e.g., genetically modified neurons derived from pluripotent stem cells) and/or genetically modified neural progenitor cells (e.g., genetically modified neural progenitor cells derived from pluripotent stem cells).

In variants leveraging modified cells, the set of cells 320 can be genetically modified by transfecting cells with a light-sensitive protein (e.g., using a virus with a plasmid and capsid) that acts as an optogenetic actuator (e.g., optogenetic effector) and/or optogenetic sensor. Optogenetic actuators produce a biochemical signal (e.g., an action potential) in response to receiving light at a specific wavelength; optogenetic sensors produce light at a specific wavelength based on (e.g., proportional to) a state of the cell (e.g., a concentration of a given molecule). However, optogenetic actuators and optogenetic sensors can be otherwise defined. In variants, optogenetic sensors can be coupled to optogenetic actuators in a cell (e.g., a fusion construct) such that when the optogenetic actuator receives a first wavelength of light the optogenetic sensor is activated to can emit a second wavelength of light based on the cell state.

The virus used to transfect the set of cells 320 can optionally be targeted to a specific cell type (e.g., general soma cells, neurons, neural progenitor cells, stem cells, etc.). The capsid can be an adeno-associated virus capsid (e.g., AAV2.7M8) and/or any other suitable capsid. The plasmid can be an opsin, a fluorescent biosensor protein, and/or any other suitable plasmid. Opsin examples include: CheRiff, ChroMD, ChroME, ChroME2S, ChRmine (e.g., ChRmine-mScarlet), ChrimsonR (e.g., including red-shifted variants), ReachR, and/or any other opsin. In an illustrative example, the set of cells 320 can be transfected using: AAV2.7m8 hSyn1-ChRmine-Kv2.1-WPRE. Fluorescent biosensor protein examples include: GCaMP8s, GCaMP8m, jRGecola, YCaMP, iGECI, and/or any other suitable protein. The opsin can be activated by blue light, green light, red light, and/or any other wavelength. Different plasmids (e.g., with different wavelength sensitivities) can optionally be used for content transfer (e.g., input), sensing, (e.g., cell monitoring), and/or sensing activation. Different plasmids can optionally be used for different types of content (e.g., different communication modalities). However, any optogenetic method can be implemented.

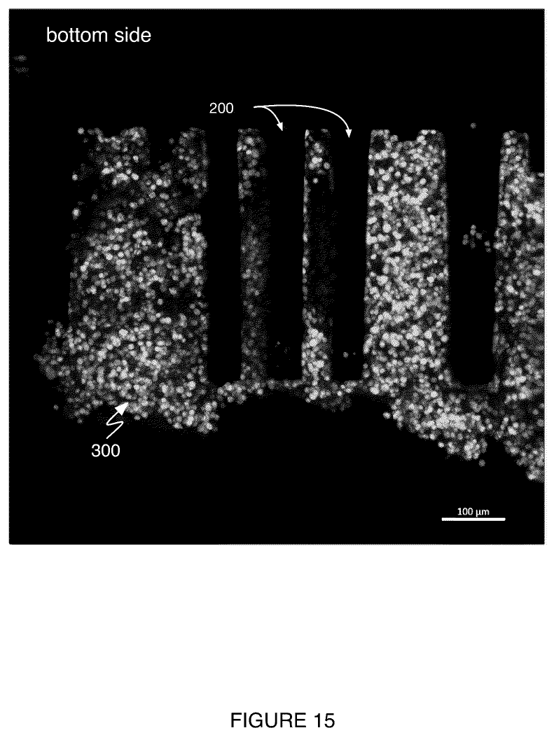

The set of cells 320 are preferably seeded between scaffolds in the set of scaffolds 200 (e.g., in the trenches between the fins), but can be otherwise integrated into the system 10. An example is shown in FIG. 15. For example, the set of cells 320 can be seeded between each pair of adjacent scaffolds in the set of scaffolds 200. In an example, the system 10 can include: a set of cells 320 (e.g., genetically modified cells), a set of scaffolds 200, wherein the set of cells 320 are seeded between adjacent pairs of scaffolds in the set of scaffolds 200; and a controller 100 communicatively connected to the set of scaffolds 200. In a specific example, each scaffold in the set of scaffolds 200 includes: an array of recording electrodes configured to receive signals from the set of cells 320; and an array of μLEDs configured to transmit light signals to the set of cells 320 based on control instructions; wherein the controller 100 is configured to: receive data from the array of recording electrodes; and determine control instructions for the array of μLEDs.

There is preferably greater than a 1:1 ratio of cells to signal elements (e.g., at least 2:1, at least 3:1, etc.), but can alternatively be a 1:1 ratio or less than a 1:1 ratio. For example, each signal element can interface with (e.g., map to) greater than 1 cell in the set of cells 320. The set of cells 320 are preferably distributed between scaffolds in multiple layers (e.g., 2-10 layers, 10-20 layers, more than 20 layers, etc.), but can alternatively be distributed in a single layer. In an example, the number of cells between each pair of scaffolds (e.g., within a trench) can be between 10-1million or any range or value therebetween (e.g., at least 100, at least 1k; at least 5k; at least 10k, at least 20k, etc.). In an example, the total number of cells across the set of scaffolds 200 can be between 1000-10million or any range or value therebetween (e.g., 0.5million-6million). In an example, the cell seeding density can be between 100 cells per mm³-500,000 cells per mm³, or any range or value therebetween (at least 500 cells per mm³; at least 1000 cells per mm³; at least 5000 cells per mm³; at least 10,000 cells per mm³; at least 50,000 cells per mm³, at least 100,000 cells per mm³, etc.). In an illustrative example, the cell seeding density can be similar to the cell density of native brain tissue.

The cell support 340 can function to: retain the bodies of the set of cells 320 within trenches in the set of scaffolds 200, support projections from the set of cells 320 into the native tissue, provide an interface between the set of cells 320 and the native tissue, diffuse molecules (e.g., nutrients) between native tissue and the set of cells 320, and/or perform other functions. In a specific example, the cell support 340 (e.g., gel) can be configured to support growth of axons and/or dendrites of the set of cells 320 out of the cell support 340. The cell support 340 is preferably a gel (e.g., a hydrogel), but can additionally or alternatively include any other suitable material. The cell support 340 material can include alginate, any biorthogonal hydrogel, and/or any other gel material. The cell support 340 is preferably fully transparent, but can alternatively be partially transparent (e.g., translucent) or non-transparent. The cell support 340 is preferably porous (e.g., microporous), but can alternatively be non-porous. The cell support 340 can optionally include growth factors, differentiation factors, small molecules, proteins, angiogenesis factors, extracellular matrix, nutrients, other chemicals, and/or other components. The cell support 340 can be positioned: between adjacent pairs of scaffolds in the set of scaffolds 200 (e.g., within the trenches), as a capping layer at the proximal end of the set of scaffolds 200, and/or otherwise positioned. The cell support 340 can optionally be adhered to the set of scaffolds 200 using: adhesive, polymerization, cross-linking, and/or any other bonding or other adhesion methods. However, any other cell support 340 can be used.

The set of cells 320 is preferably loaded into the set of scaffolds 200 (e.g., into the trenches) prior to implantation of the system 10, but can alternatively be loaded at any other time. In a specific example, the set of cells 320 are loaded into the set of scaffolds 200 with a liquid, unpolymerized cell support 340 (e.g., hydrogel), wherein the cell support 340 is polymerized (e.g., using calcium and/or any other polymerization factor) after loading. In a specific example, the set of scaffolds 200 can be capped with additional the cell support 340 (e.g., hydrogel).

In a first variant, neurons (e.g., post-differentiation) are loaded into the set of scaffolds 200 (e.g., into the trenches). In a specific example, the set of cells 320 can be loaded into the set of scaffolds 200 by performing one or more of: placing the set of scaffolds 200 in a loading liquid (e.g., the loading liquid occupies the trenches between scaffolds), placing the set of cells 320 in a loading fixture, centrifuging cells from the loading fixture into the trenches between the scaffolds (e.g., the cells adhere to the scaffolds), washing all or a portion of the set of scaffolds 200, and/or any other suitable steps.

In a second variant, stem cells and/or neural progenitor cells are first loaded into the set of scaffolds 200 and then differentiated. Differentiation can occur before or after implantation. In variants, differentiating after loading can reduce stress on the set of cells. In an example, the stem cells and/or neural progenitor cells can grow into (polymerized or unpolymerized) cell support 340 in the set of scaffolds 200. In a first specific example, differentiation factors can be added to the cell support 340 to induce or complete differentiation of the cells. In a second specific example, differentiation of the set of cells 320 can be induced or completed by implanting the system 10 in or on the brain. However, the set of cells 320 can be otherwise loaded.

However, the tissue interface 300 can be otherwise configured.

Implanting the system 10 in a user can optionally include one or more of: performing an incision on the skull, performing a craniectomy, resecting the dura, partially or fully ablating the pia mater (e.g., ablating using a laser; mechanically ablating using a blade; etc.), positioning the proximal subsystem 12 (including the proximal controller module 120, the set of scaffolds 200, and the tissue interface 300) on the brain surface (e.g., with pressure) such that the tissue interface 300 is in contact with the brain surface, folding the dura over all or a portion of the proximal subsystem 12, anchoring and/or sealing the proximal subsystem 12 to tissue, positioning the distal subsystem 14, anchoring and/or sealing the distal subsystem 14 to tissue, suturing skin around or over the distal subsystem 14, and/or any other suitable implantation steps.

The system 10 can optionally include a power source, which functions to power the controller 100 (e.g., the proximal controller module 120, the distal controller module 140, etc.), the set of scaffolds 200, and/or any other implanted system components. In a first variant, the power source is implanted (e.g., a battery implant). In a second variant, the power source (or a component of the power source) is remote to the implanted system components. For example, the power source can include a power transmitter component remote to the implanted system components (e.g., coupled to the external device 400, coupled to a separate device, etc.) as well as a power receiver component connected to one or more implanted system components (e.g., coupled to the controller 100). In specific examples, power receiver components can include: a magnetic coil, a magnetoresistive film, and/or other power receiver components that can interface with an external induction power source. In examples, the power source can provide power to the implanted system components via induction, IR, RF, and/or any other remote power method. However, the power source can be otherwise configured.

In an example, the power consumption of the implanted system components can be between 10 mW-100 mW or any range or value therebetween (e.g., 30 mW-35 mW, less than 50 mW, etc.).

The system 10 can optionally include or be used with an external device 400, which can function to: determine the control instructions (e.g., excitation parameters), receive data (e.g., from the controller 100), process data, and/or perform any other functions. Examples of external devices that can be used include: a user device, a processing system (e.g., remote processing system), and/or any other suitable external device. The external device 400 can optionally include one or more of: communication elements configured to communicate with the controller 100 (e.g., with the distal controller module 140), processing system, sensors, power components, and/or any other suitable components. Information transmitted by the external device 400 can include: content (e.g., processed or unprocessed content), control instructions (e.g., calculated based on the content), and/or any other information. Information received by the external device 400 can include: data (e.g., cell states measured by the sensor elements), controller measurements (e.g., controller states measured by the controller sensor), and/or any other information.

However, the external device 400 can be otherwise configured.

In variants, the system 10 can include systems and methods as described in Appendix A, which is incorporated in its entirety by this reference. In variants, the system 10 can include systems and methods disclosed in: “A thin-film optogenetic visual prosthesis” (Knudsen E B, Zappitelli K, Brown J, Reeder J, Smith K S, Rostov M, Choi J, Rochford A, Slager N, Miura S K, Rodgers K, Reed A, Israeli Y R L, Shiraga S, Seo K J, Wolin C, Dawson P, Eltaeb M, Dasgupta A, Chong P, Charles S, Stewart J M, Silva R A, Kim T, Kong Y, Mardinly A R, Hodak M. 2023. bioRxiv doi: 10.1101/2023.01.31.526482), which is incorporated in its entirety by this reference.

However, the system 10 can be otherwise configured.

### 5. Specific Examples

A numbered list of specific examples of the technology described herein are provided below. A person of skill in the art will recognize that the scope of the technology is not limited to and/or by these specific examples.

Specific Example 1. A system configured to be implanted on a surface of a brain of a user, comprising: a set of genetically modified cells; a set of scaffolds, wherein the set of genetically modified cells are seeded between adjacent pairs of scaffolds in the set of scaffolds, wherein each scaffold in the set of scaffolds comprises: an array of recording electrodes configured to receive signals from the set of genetically modified cells; and an array of μLEDs configured to transmit light signals to the set of genetically modified cells based on control instructions; and a controller communicatively connected to the set of scaffolds, wherein the controller is configured to: receive data from the array of recording electrodes; and determine control instructions for the array of μLEDs.

Specific Example 2. The system of Specific Example 1, wherein the genetically modified cells comprise genetically modified neurons derived from pluripotent stem cells.

Specific Example 3. The system of any of Specific Examples 1 or 2, wherein the system further comprises a gel between adjacent pairs of scaffolds in the set of scaffolds, wherein the gel is configured to retain the genetically modified cells.

Specific Example 4. The system of Specific Example 3, wherein the gel is configured to support growth of axons of the genetically modified cells out of the gel.

Specific Example 5. The system of any of Specific Examples 1-4, further comprising an anchor configured to secure the set of scaffolds to the surface of the brain of the user.

Specific Example 6. The system of any of Specific Examples 1-5, wherein the controller comprises a proximal controller module and a distal controller module, wherein the proximal controller module is coupled to the set of scaffolds, and wherein the distal controller module is anchored to the skull of the user, wherein the distal controller module is communicatively coupled to an external device.

Specific Example 7. The system of Specific Example 6, further comprising a flexible connector connecting the proximal controller module to the distal controller module.

Specific Example 8. The system of any of Specific Examples 1-7, wherein, for each scaffold in the set of scaffolds, the array of recording electrodes and the array of μLEDs are located on opposing faces of the scaffold.

Specific Example 9. The system of any of Specific Examples 1-8, wherein the genetically modified cells are transfected with a gene for a light-sensitive protein, wherein the genetically modified cells produce biochemical signals in response to receiving the light signals.

Specific Example 10. The system of any of Specific Examples 1-9, wherein at least 100 genetically modified cells are seeded between each pair of adjacent scaffolds.

Specific Example 11. The system of any of Specific Examples 1-10, wherein the set of scaffolds comprises at least 10 scaffolds.

Specific Example 12. A system configured to be implanted on a surface of a brain of a user, comprising: a first scaffold comprising an array of μLEDs on a first face of the first scaffold; a second scaffold comprising an array of recording electrodes on a first face of the second scaffold; a spacer separating the second scaffold from the first scaffold, the spacer positioned between the first face of the first scaffold and the first face of the second scaffold; a gel positioned between the array of μLEDs and the array of recording electrodes; and a set of genetically modified cells retained within the gel, wherein the array of recording electrodes is configured to receive signals from the set of genetically modified cells, wherein the array of μLEDs is configured to transmit light signals to the set of genetically modified cells.

Specific Example 13. The system of Specific Example 12, wherein the spacer comprises a first raised conductive element in contact with the first face of the first scaffold and second raised conductive element in contact with the first face of the second scaffold, the system further comprising a connector connecting the first raised conductive element to the second raised conductive element.

Specific Example 14. The system of any of Specific Examples 12-13, wherein the first scaffold further comprises a second array of recording electrodes on a second face of the first scaffold, wherein the second face of the first scaffold is opposite the first face of the first scaffold, and wherein the second scaffold further comprises a second array of μLEDs on a second face of the second scaffold, wherein the second face of the second scaffold is opposite the first face of the second scaffold.

Specific Example 15. The system of any of Specific Examples 12-14, wherein the set of genetically modified cells comprise axons extending out of the gel, wherein the axons interface with native neurons in the brain of the user.

Specific Example 16. The system of any of Specific Examples 12-15, wherein a thickness of the spacer is at least 100 μm.

Specific Example 17. The system of any of Specific Examples 12-16, wherein the genetically modified cells are transfected with a gene for a light-sensitive protein, wherein the genetically modified cells produce biochemical signals in response to receiving the light signals.

Specific Example 18. The system of Specific Example 17, wherein the set of cells comprise hypoimmune cells, wherein the set of cells are further transfected with a killswitch gene.

Specific Example 19. The system of any of Specific Examples 12-18, wherein the array of μLEDs comprises at least 100 μLEDs.

Specific Example 20. The system of any of Specific Examples 19, wherein the array of recording electrodes comprises at least 100 recording electrodes.

As used herein, “substantially” or other words of approximation (e.g., “about,” “approximately,” etc.) can be within a predetermined error threshold or tolerance of a metric, component, or other reference (e.g., within +/−0.001%, +/−0.01%, +/−0.1%, +/−1%, +/−2%, +/−5%, +/−10%, +/−15%, +/−20%, +/−30%, any range or value therein, of a reference).

All references cited herein are incorporated by reference in their entirety, except to the extent that the incorporated material is inconsistent with the express disclosure herein, in which case the language in this disclosure controls.

Different subsystems and/or modules discussed above can be operated and controlled by the same or different entities. In the latter variants, different subsystems can communicate via: APIs (e.g., using API requests and responses, API keys, etc.), requests, and/or other communication channels. Communications between systems can be encrypted (e.g., using symmetric or asymmetric keys), signed, and/or otherwise authenticated or authorized.

Alternative embodiments implement the above methods and/or processing modules in non-transitory computer-readable media, storing computer-readable instructions that, when executed by a processing system, cause the processing system to perform the method(s) discussed herein. The instructions can be executed by computer-executable components integrated with the computer-readable medium and/or processing system. The computer-readable medium may include any suitable computer readable media such as RAMs, ROMs, flash memory, EEPROMs, optical devices (CD or DVD), hard drives, floppy drives, non-transitory computer readable media, or any suitable device. The computer-executable component can include a computing system and/or processing system (e.g., including one or more collocated or distributed, remote or local processors) connected to the non-transitory computer-readable medium, such as CPUs, GPUs, TPUS, microprocessors, or ASICs, but the instructions can alternatively or additionally be executed by any suitable dedicated hardware device.

Embodiments of the system and/or method can include every combination and permutation of the various system components and the various method processes, wherein one or more instances of the method and/or processes described herein can be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method can be used with, in addition to, in lieu of, or otherwise integrated with all or a portion of the systems and/or methods disclosed in the applications mentioned above, each of which are incorporated in their entirety by this reference.

As a person skilled in the art will recognize from the previous detailed description and from the figures and claims, modifications and changes can be made to the preferred embodiments of the invention without departing from the scope of this invention defined in the following claims.

We claim:

1. A system configured to be implanted on a surface of a brain of a user, comprising:
a set of genetically modified cells;
a set of scaffolds, wherein the set of genetically modified cells are seeded between adjacent pairs of scaffolds in the set of scaffolds, wherein each scaffold in the set of scaffolds comprises:
an array of recording electrodes configured to receive signals from the set of genetically modified cells; and
an array of μLEDs configured to transmit light signals to the set of genetically modified cells based on control instructions; and
a controller communicatively connected to the set of scaffolds, wherein the controller is configured to:
receive data from the array of recording electrodes; and
determine control instructions for the array of μLEDs.

2. The system of claim 1, wherein the genetically modified cells comprise genetically modified neurons derived from pluripotent stem cells.

3. The system of claim 1, wherein the system further comprises a gel between adjacent pairs of scaffolds in the set of scaffolds, wherein the gel is configured to retain the genetically modified cells.

4. The system of claim 3, wherein the gel is configured to support growth of axons of the genetically modified cells out of the gel.

5. The system of claim 1, further comprising an anchor configured to secure the set of scaffolds to the surface of the brain of the user.

6. The system of claim 1, wherein the controller comprises a proximal controller module and a distal controller module, wherein the proximal controller module is coupled to the set of scaffolds, and wherein the distal controller module is anchored to the skull of the user, wherein the distal controller module is communicatively coupled to an external device.

7. The system of claim 6, further comprising a flexible connector connecting the proximal controller module to the distal controller module.

8. The system of claim 1, wherein, for each scaffold in the set of scaffolds, the array of recording electrodes and the array of μLEDs are located on opposing faces of the scaffold.

9. The system of claim 1, wherein the genetically modified cells are transfected with a gene for a light-sensitive protein, wherein the genetically modified cells produce biochemical signals in response to receiving the light signals.

10. The system of claim 1, wherein at least 100 genetically modified cells are seeded between each pair of adjacent scaffolds.

11. The system of claim 1, wherein the set of scaffolds comprises at least 10 scaffolds.

12. A system configured to be implanted on a surface of a brain of a user, comprising:
a first scaffold comprising an array of μLEDs on a first face of the first scaffold;
a second scaffold comprising an array of recording electrodes on a first face of the second scaffold;
a spacer separating the second scaffold from the first scaffold, the spacer positioned between the first face of the first scaffold and the first face of the second scaffold;
a gel positioned between the array of μLEDs and the array of recording electrodes; and
a set of genetically modified cells retained within the gel, wherein the array of recording electrodes is configured to receive signals from the set of genetically modified cells, wherein the array of μLEDs is configured to transmit light signals to the set of genetically modified cells.

13. The system of claim 12, wherein the spacer comprises a first raised conductive element in contact with the first face of the first scaffold and second raised conductive element in contact with the first face of the second scaffold, the system further comprising a connector connecting the first raised conductive element to the second raised conductive element.

14. The system of claim 12, wherein the first scaffold further comprises a second array of recording electrodes on a second face of the first scaffold, wherein the second face of the first scaffold is opposite the first face of the first scaffold, and wherein the second scaffold further comprises a second array of μLEDs on a second face of the second scaffold, wherein the second face of the second scaffold is opposite the first face of the second scaffold.

15. The system of claim 12, wherein the set of genetically modified cells comprise axons extending out of the gel, wherein the axons interface with native neurons in the brain of the user.

16. The system of claim 12, wherein a thickness of the spacer is at least 100 μm.

17. The system of claim 12, wherein the genetically modified cells are transfected with a gene for a light-sensitive protein, wherein the genetically modified cells produce biochemical signals in response to receiving the light signals.

18. The system of claim 17, wherein the set of cells comprise hypoimmune cells, wherein the set of cells are further transfected with a killswitch gene.

19. The system of claim 12, wherein the array of μLEDs comprises at least 100 μLEDs.

20. The system of claim 12, wherein the array of recording electrodes comprises at least 100 recording electrodes.
